(19) United States
(12) Patent Application Publication
(10) Pub. No.: US 2025/0381332 A1
(43) Pub. Date: Dec. 18, 2025

(54) INTERFACE DEVICE FOR MEDICAL DEVICES AND METHOD

(71) Applicant: Fresenius Medical Care Deutschland GmbH, Bad Homburg (DE)

(72) Inventor: Stefan Wegner, Werneck (DE)

(73) Assignee: Fresenius Medical Care Deutschland GmbH, Bad Homburg (DE)

(21) Appl. No.: 19/233,834

(22) Filed: Jun. 10, 2025

(30) Foreign Application Priority Data
Jun. 17, 2024 (EP) 24182489

(51) Int. Cl. A61M 1/28; G16H 40/67

(52) U.S. Cl. CPC A61M 1/282; G16H 40/67

(57) ABSTRACT

An interface device for data exchange of medical devices with remote devices via networks ensures that the data transfer from the medical device to the interface device can be read out reliably and that the data exchange with the at least one remote device is robust with respect to undesired data exchange. The medical devices may include, for example, hemodialysis (HD) or peritoneal dialysis (PD) treatment devices.

### CROSS-REFERENCE TO RELATED APPLICATION

Priority is claimed to European Patent Application No. EP 24182489, filed on Jun. 17, 2024, the entire disclosure of which is hereby incorporated by reference herein.

### TECHNICAL FIELD

The present invention is in the field of medical technology, in particular in the field of interface devices for data exchange of medical devices with remote devices via networks and corresponding methods for this purpose.

### BACKGROUND

Large amounts of data accumulate during the use of medical devices, in particular those which are directly used for treating patients. These data comprise data relating to operating statuses of the medical device or further devices connected thereto, and also data relating to a patient treated using the medical device. Such data are used for monitoring, controlling, and/or regulating the medical device or a medical treatment carried out thereby.

The data integrity and security are accordingly of particular importance for such data, since the security and the treatment success of the patient depends thereon.

In the past, the use of such data was restricted to the medical device itself and this was used, for example, to display operating statuses or treatment statuses on a respective display such as a display screen. Such medical devices often only have restricted data exchange options such as serial data interfaces, for example, RS-232 interfaces, which are not configured for data exchange using modern network standards for data exchange with, for example, hospital networks or the Internet.

### SUMMARY

In an exemplary embodiment, the present disclosure provides an interface device. The interface device includes: at least one integrated circuit configured to be controlled by software; at least one storage device configured to store the software; one or more first interfaces configured for data exchange between a medical device and the interface device; and one or more second interfaces configured for data exchange between the interface device and at least one remote device via at least one network. The at least one integrated circuit is configured to supervise the data exchanges. The at least one storage device is configured such that the software stored in the at least one storage device is updatable. The one or more first interfaces are secured by the interface device such that updates to the software stored in the at least one storage device cannot influence data transfers via the one or more first interfaces.

In a further exemplary embodiment, the present disclosure provides a method. The method includes: controlling, via software, a software-controlled interface device, wherein the software is stored in at least one storage device of the software-controlled interface device; updating the software; exchanging, via one or more first interfaces of the software-controlled interface device, data between a medical device and the software-controlled interface device via at least one first network; and exchanging, via one or more second interfaces of the software-controlled interface device, data between the software-controlled interface device and at least one remote device via at least one second network. The one or more first interfaces of the software-controlled interface device are secured such that updates to the software stored in the at least one storage device cannot influence data transfers via the one or more first interfaces.

In yet another further exemplary embodiment, the present disclosure provides one or more non-transitory computer-readable mediums having processor-executable instructions stored thereon. The processor-executable instructions, when executed, facilitate performance of the following: controlling, via software, a software-controlled interface device, wherein the software is stored in at least one storage device of the software-controlled interface device; updating the software; exchanging, via one or more first interfaces of the software-controlled interface device, data between a medical device and the software-controlled interface device via at least one first network; and exchanging, via one or more second interfaces of the software-controlled interface device, data between the software-controlled interface device and at least one remote device via at least one second network. The one or more first interfaces of the software-controlled interface device are secured such that updates to the software stored in the at least one storage device cannot influence data transfers via the one or more first interfaces.

### BRIEF DESCRIPTION OF THE DRAWINGS

Exemplary embodiments of the present application will be described in even greater detail below based on the exemplary figures. The application is not limited to the exemplary embodiments. All features described and/or illustrated herein can be used alone or combined in different combinations in embodiments of the application. Features and advantages of various embodiments of the present application will become apparent by reading the following detailed description with reference to the attached drawings which illustrate the following:

FIG. 1 shows an exemplary schematic view of an interface device 100 according to the invention and a typical use of the interface device 100 according to the invention; and

FIG. 2 shows a further schematic view of an interface device 100 according to the invention.

### DETAILED DESCRIPTION

Exemplary embodiments provide for incorporating medical devices into modern networks and ensuring the security and data integrity of the data of the medical device in this case and in particular protecting the data from attacks (such as cyberattacks) from these networks.

Exemplary embodiments of the present invention provide for receiving data from a medical device and making these data accessible to at least one remote device via at least one network, where it is ensured that the data of the medical device can be read reliably and the data exchange with the at least one remote device is robust with respect to undesired data exchange.

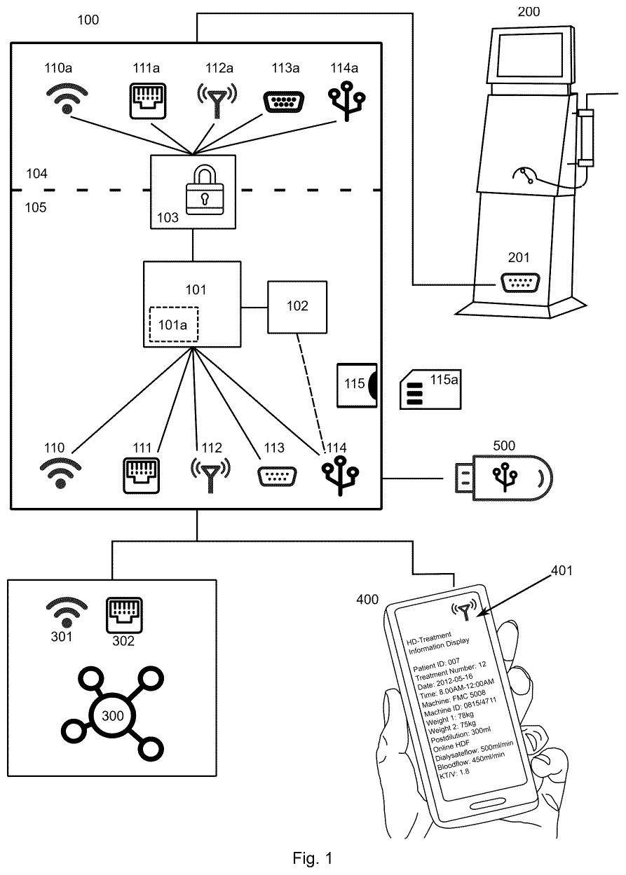

The interface device 100 shown in FIG. 1 comprises an integrated circuit 101, which can be embodied, for example, as a central processing unit (CPU) or microcontroller. The integrated circuit is programmed by software and configured to control the interface device 100 in this way. The software which is used for this purpose is stored in the storage device 102. The storage device 102 can be embodied, for example, as an erasable programmable read-only memory (EPROM), electrically erasable programmable read-only memory (EEPROM), flash memory storage device, or another electronic memory which is rewritable and the storage content of which is retained in a nonvolatile manner.

The interface device 100 comprises a plurality of interfaces which are configured for data exchange with at least one remote device via at least one network. These interfaces are embodied by way of example in FIG. 1 as a wireless local area network (WLAN) interface 110, local area network (LAN) interface 111, mobile wireless interface 112, serial interface 113, embodied, for example, as a RS-232 interface, and a further serial interface 114, embodied, for example, as a Universal Serial Bus (USB) interface. A further Inter-Integrated Circuit (I2C) interface may be provided as well. An interface for receiving memory cards, such as the Secure Digital (SD) slot 115 for receiving an SD memory card 115a, can likewise be present. This can also be used in particular for updating the software which is stored in the storage device 102, in that the updated software is present on a memory card.

Remote devices can be, for example, computers, which exchange data via a clinical network, mobile computers, such as laptops or tablet computers, which are assigned to a technician, for example, and exchange data via WLAN, USB, Bluetooth, or radiofrequency identification (RFID), or mobile wireless devices such as smartphones, which typically communicate via WLAN or mobile wireless.

A clinical network having devices 300 is symbolized by way of example in FIG. 1, which exchanges data via WLAN 301 or LAN 302. Furthermore, a mobile terminal is shown in FIG. 1, which is embodied as a smartphone 400 and which exchanges data via a mobile wireless connection 401.

Furthermore, FIG. 1 shows a USB storage device 500, which can exchange data with the interface device 100 via a corresponding interface 114. This is symbolized in FIG. 1 by the dashed line from the USB interface 114 to the storage device 102. In this way, the software which controls the integrated circuit 101 can also be stored in the storage device 102. However, it is also possible to save the software on the storage device 102 via one of the other interfaces 110 to 113 and 115.

The interface device 100 furthermore has at least one interface 110a, 111a, 112a, 113a, and/or 114a, which are embodied corresponding to the interfaces 110 to 114. In principle, the interface device can comprise any suitable data interface which is configured to exchange data with a remote device 300, 400 and/or a medical device 200 or receive data from there. Such interfaces are not restricted to serial interfaces, parallel interfaces, RS-232 interfaces, I2C interfaces, USB interfaces, LAN interfaces, WLAN interfaces, RFID interfaces, near field communication (NFC) interfaces, Bluetooth interfaces, and infrared interfaces, but can be embodied in this way.

In exemplary embodiments, interfaces which have galvanic decoupling are used. This isolates the electrical systems of the devices participating in the data exchange such that no current is exchanged between them. Such interfaces can comprise, for example, optocouplers or isolating transformers.

These interfaces are used for data exchange with a medical device 200, which is embodied by way of example as a hemodialysis device in FIG. 1. A hemodialysis device is a blood treatment device. Other blood treatment devices which likewise can exchange data with the interface device 100 are devices for peritoneal dialysis, hemofiltration, hemodiafiltration, or plasmapheresis or also devices for extracorporeal cardiac and pulmonary assistance. In principle, any medical device is capable of exchanging data with the interface device 100 if it has an interface configured correspondingly thereto.

The software which programs the integrated circuit 101 to control the interface device 100 can cause it to execute a variety of methods and therefore configure the interface device 100. These methods can comprise: checking for data security the data which arrive from one or more of the interfaces 110 to 114 from a remote device 300, 400. This check comprises, for example, querying the origin of the data via the IP addresses or hardware addresses (media access control (MAC) addresses) sent with the data, checking data contents for harmful content via corresponding software stored in the interface device such as software for defending from malware, ransomware, or computer viruses, checking for denial-of-service attacks, etc. In general, it is possible according to the invention, by updating the software of the interface device 100, to program it to defend from all possible known impermissible data traffic.

Furthermore, data which arrive from one or more of the interfaces 110 to 114 from a remote device can be filtered. This can comprise filtering the data to filter out specific content, such as patient data or data relating to the medical device 200, from the data and to prepare the data to be passed on to the medical device 200 and/or to a remote device 300, 400.

Furthermore, filtered or unfiltered data from multiple remote devices 300, 400 can be merged, thus bundled into data packets. These data packets can have a specific proprietary data format which can be processed by the devices 300, 400 or also the medical device 200 by corresponding programming.

In one embodiment, the interface device is configured to encrypt data of the medical device 200 and/or to decrypt encrypted data of the remote devices 300, 400. These encryption and decryption methods are freely selectable and updatable by the updatable software.

In an exemplary embodiment, the interface device 100 is configured to exchange data with the medical device 200 via a serial interface, in particular an RS-232 interface 113a or a USB interface 114a.

In a further embodiment, the interface device comprises a data transformation unit 101a to convert data from the data exchange between the medical device 200 and at least one remote device 300, 400 from a first into a second data format and/or vice versa. This data transformation unit 101a is configured to convert the data formats of the participating devices into the respective other one. Medical devices, in particular those which are already old, often operate using proprietary data formats which are not compatible with currently typical data formats within the clinical environment such as HL7 (Health Level 7). The data transformation unit 101a is accordingly programmed or configured to convert the data of the participating devices into the format which is compatible in each case for the individual device to which the data are sent. The data transformation unit 101a can be embodied as software which is executed by the integrated circuit 101. The data transformation unit 101a can also be a dedicated application-specific integrated circuit (ASIC), which is configured for this task. According to this embodiment, the data transformation unit 101a in FIG. 1 is shown by dashed lines within the integrated circuit. An embodiment as a dedicated integrated circuit which operates independently of the integrated circuit 101 is likewise possible.

In preferred embodiments, the interface device 100 is configured to keep the data transfer from the medical device 200 to the interface device 100 not able to be influenced by updated software which controls the integrated circuit 101. This can be implemented on the basis of the updated software itself, in that this updated software does not contain code which would change the corresponding data transfer from the medical device 200 to the interface device 100.

A further possibility for making the data transfer from the medical device 200 to the interface device 100 not able to be influenced by updated software is to embody the interfaces 110a to 114a as non-programmable interfaces. These are generally embodied as corresponding integrated circuits in conjunction with the mechanically embodied electrical connections such as terminal strips or sockets.

In a further embodiment, it can be provided that the interfaces 110a to 114a are made programmable for a first configuration for data exchange with a specific medical device 200 and are blocked for further programming after completed configuration. This can take place, for example, in that electrical conductor tracks which are required for programming the interfaces are destroyed mechanically or by overcurrent (so-called fusing). Renewed programming is prevented in this way. One alternative to destroying the corresponding conductor tracks is to interrupt the electrical connections so they can be reestablished, for example, by removing jumpers from plug contacts, by which an electrical connection is interrupted, but can be reestablished if needed by plugging on the jumper again. Further programming is obstructed in this way.

In FIG. 1, this inability to influence the data transfer from the medical device 200 to the interface device 100 is symbolized by the box 103 with a lock drawn on. This is not to be understood as an explicit device, but rather symbolizes the isolation of the data handling by the interface device in an area 104, in which data are always received from the medical device 200 in the same manner that cannot be influenced, and in an area 104, in which a data exchange with remote devices takes place, in which this data exchange can be influenced by updated software that controls the integrated circuit 101.

This isolation ensures that data from the medical device always reach the interface device 100 in the same manner and unable to be influenced by the programming thereof. This is advantageous in the medical field, since these data are of enormous importance for the treatment success and the security of a patient who is treated using the medical device. It is therefore advantageous that these data are received in a manner which is reliable and cannot be influenced. However, it is also advantageous to check data received from remote devices 300, 400 for data security, and to filter these data, and possibly to decrypt and/or bundle them. Since the remote devices 300, 400 can be of a variety of natures and additionally are subject to continuous technical development, in the same way that the challenges with respect to data security continuously develop further, it is advantageous to make the data interface adaptable in this regard. This is made possible by the option of updating the software which controls the integrated circuit 101. Accordingly, a secured data transfer from the medical device 200 to the interface device 100 and a secure data exchange with remote devices 300, 400 is enabled by the interface device 100.

The underlying method according to the invention for controlling the software-controlled interface device 100 comprises the software being able to be stored repeatedly in the interface device 100, so that this can be updated, and the interface device 100 is configured for data exchange between a medical device 200 and at least one remote device 300, 400 via at least one network interface 110 to 114, and updating the software does not influence a data transfer from the medical device 200 to the interface device 100.

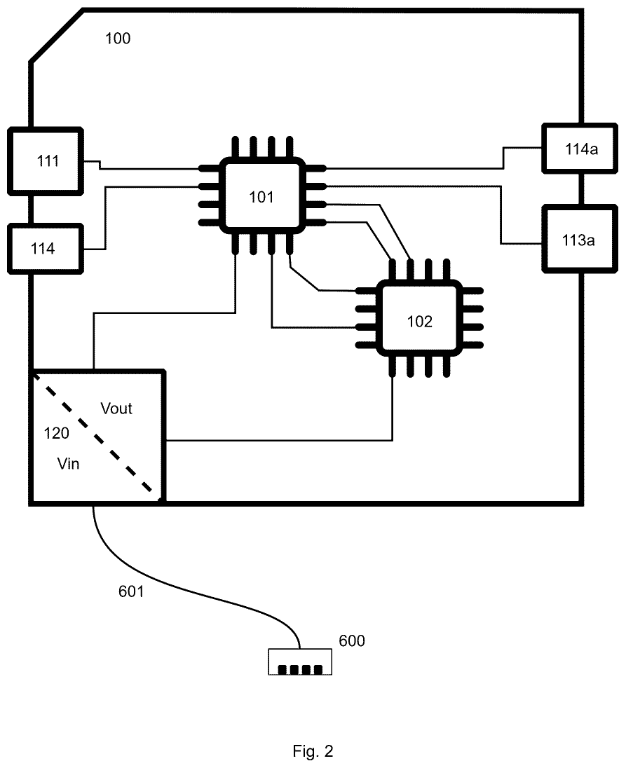

In a further embodiment, the interface device is supplied with electric energy by the medical device 200. This is schematically shown in FIG. 2.

The interface device 100 according to FIG. 2 shows the integrated circuit 101, and the storage device 102, which are electrically connected via symbolized conductor tracks to one another and to a device 120 for supplying the interface device 100 with electric energy. This device 120 is configured to be electrically connected to one or more voltage sources of the medical device 200. The device 120 is furthermore configured to generate one or more output voltages Vout, which are used to supply the interface device with electric energy, from the one or more voltage(s), which are therefore used as input voltage(s) Vin. Such a device 120 can comprise an AC/DC converter for converting AC voltage into DC voltage and/or a DC/DC converter for converting DC voltage into a different DC voltage. A person skilled in the art knows a variety of circuits, in particular also integrated circuits, which manage these tasks.

The connecting of the device 120 to one or more voltage sources of the medical device 200 can be implemented via an adapted electric line 601, and a plug connection 600 adapted to the conditions of the medical device. Such a plug connection can be, for example, a socket strip or a plug strip, or any electromechanical connection configured for connecting to the corresponding counterpart of the medical device.

In a further embodiment, the interface device 100 comprises a device for installing the interface device 100 inside the medical device 200. This device is adapted to the conditions of the medical device and can comprise, for example, a correspondingly embodied busbar, a catch device for engaging in correspondingly provided catch points in the medical device, screw receptacles, screw connections, etc.

In a further embodiment, the interface device 100 is protected from harmful electromagnetic radiation. This is particularly important in particular during the installation of the interface device 100 in the medical device 200, because interference due to electromagnetic radiation can endanger the treatment of the patient if it results in malfunctions of the medical device. For this purpose, the interface device can have shielding plates or shielding paints, which shield the parts of the interface device from emitting or receiving electromagnetic interference radiation. It is likewise possible to install the complete interface device 100 inside a shielded housing.

The software which programs the integrated circuit 101 to control the interface device 100 as described can be provided in a software product, which can also be embodied in particular as a machine-readable medium, such as a USB stick or SD card.

It will be appreciated that the execution of the various machine-implemented processes and steps described herein may occur via the execution, by one or more respective processors, of processor-executable instructions stored on one or more tangible, non-transitory computer-readable mediums (such as random access memory (RAM), read-only memory (ROM), programmable read-only memory (PROM), and/or another electronic memory mechanism). Thus, for example, operations performed by various components as discussed herein may be carried out according to instructions stored on and/or applications installed on one or more respective computing devices.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. An interface device, comprising:
at least one integrated circuit configured to be controlled by software;
at least one storage device configured to store the software;
one or more first interfaces configured for data exchange between a medical device and the interface device; and
one or more second interfaces configured for data exchange between the interface device and at least one remote device via at least one network;
wherein the at least one integrated circuit is configured to supervise the data exchanges;
wherein the at least one storage device is configured such that the software stored in the at least one storage device is updatable; and
wherein the one or more first interfaces are secured by the interface device such that updates to the software stored in the at least one storage device cannot influence data transfers via the one or more first interfaces.

2. The interface device according to claim 1, wherein the at least one integrated circuit is configured to convert data from a first format into a second data format.

3. The interface device according to claim 1, wherein the one or more first interfaces and the one or more second interfaces include:
one or more serial interfaces,
one or more parallel interfaces,
one or more RS-232 interfaces,
one or more Universal Serial Bus (USB) interfaces,
one or more local area network (LAN) interfaces,
one or more wireless local area network (WLAN) interfaces,
one or more radiofrequency identification (RFID) interfaces,
one or more near-field communication (NFC) interfaces,
one or more Inter-Integrated Circuit (I2C) interfaces,
one or more Bluetooth interfaces, and/or
one or more infrared interfaces.

4. The interface device according to claim 1, wherein the one or more first interfaces include a serial interface configured for data connection to the medical device.

5. The interface device according to claim 1, wherein the at least one integrated circuit is configured to:
filter data;
check data for data security; and/or
bundle data into data packets.

6. The interface device according to claim 1, wherein the at least one integrated circuit is configured to encrypt data of the medical device and/or to decrypt encrypted data of the at least one remote device.

7. The interface device according to claim 1, wherein at least one interface of the one or more first interfaces or the one or more second interfaces is a galvanically isolated interface.

8. The interface device according to claim 1, wherein the one or more second interfaces comprise a Universal Serial Bus (USB) interface or a storage card interface; and
wherein the interface device is configured to receive the software via the USB interface or via the storage card interface.

9. The interface device according to claim 1, further comprising:
a device for supplying the interface device with electric energy, wherein the device is adapted to an electric energy supply of the medical device.

10. The interface device according to claim 1, further comprising:
a device for installing the interface device inside the medical device, wherein the device is adapted to an interior of the medical device.

11. The interface device according to claim 1, wherein the interface device is installed in the medical device.

12. The interface device according to claim 1, wherein the medical device is configured to provide dialysis treatment.

13. A method, comprising:
controlling, via software, a software-controlled interface device, wherein the software is stored in at least one storage device of the software-controlled interface device;
updating the software;
exchanging, via one or more first interfaces of the software-controlled interface device, data between a medical device and the software-controlled interface device via at least one first network; and
exchanging, via one or more second interfaces of the software-controlled interface device, data between the software-controlled interface device and at least one remote device via at least one second network;
wherein the one or more first interfaces of the software-controlled interface device are secured such that updates to the software stored in the at least one storage device cannot influence data transfers via the one or more first interfaces.

14. One or more non-transitory computer-readable mediums having processor-executable instructions stored thereon, wherein the processor-executable instructions, when executed, facilitate performance of the following:
controlling, via software, a software-controlled interface device, wherein the software is stored in at least one storage device of the software-controlled interface device;
updating the software;
exchanging, via one or more first interfaces of the software-controlled interface device, data between a medical device and the software-controlled interface device via at least one first network; and
exchanging, via one or more second interfaces of the software-controlled interface device, data between the software-controlled interface device and at least one remote device via at least one second network;
wherein the one or more first interfaces of the software-controlled interface device are secured such that updates to the software stored in the at least one storage device cannot influence data transfers via the one or more first interfaces.

15. A medical system, comprising:
a medical device configured to provide a medical treatment; and
an interface device, wherein the interface device comprises:
at least one integrated circuit configured to be controlled by software;
at least one storage device configured to store the software;
one or more first interfaces configured for data exchange between the medical device and the interface device; and
one or more second interfaces configured for data exchange between the interface device and at least one remote device via at least one network;
wherein the at least one integrated circuit is configured to supervise the data exchanges;
wherein the at least one storage device is configured such that the software stored in the at least one storage device is updatable; and
wherein the one or more first interfaces are secured by the interface device such that updates to the software stored in the at least one storage device cannot influence data transfers via the one or more first interfaces.

16. The medical system according to claim 15, wherein the at least one integrated circuit is configured to:
convert data from a first format into a second data format; and/or
encrypt data of the medical device and/or to decrypt encrypted data of the at least one remote device.

17. The medical system according to claim 15, wherein the at least one integrated circuit is configured to:
filter data;
check data for data security; and/or
bundle data into data packets.

18. The medical system according to claim 15, further comprising:
a device for installing the interface device inside the medical device, wherein the device is adapted to an interior of the medical device.

19. The medical system according to claim 15, wherein the interface device is installed in the medical device.

20. The medical system according to claim 15, wherein the medical device is configured to provide dialysis treatment.
